(19) United States
(12) Patent Application Publication
(10) Pub. No.: US 2019/0374206 A1
(43) Pub. Date: Dec. 12, 2019

(54) APPARATUS, SYSTEM AND METHOD FOR DETECTING A SUBJECT'S SUSCEPTIBILITY TO INJURY

(71) Applicant: CAP CORPORATION, Calgary (CA)

(72) Inventors: Margaret Kim Adolphe, Calgary (CA); Sherwin Readiy Ganpatt, Calgary (CA)

(21) Appl. No.: 16/548,670

(22) Filed: Aug. 22, 2019

Related U.S. Application Data
(63) Continuation of application No. PCT/CA2018/050211, filed on Feb. 23, 2018.
(60) Provisional application No. 62/462,495, filed on Feb. 23, 2017.

(51) Int. Cl. A61B 9/00; A61B 5/11

(52) U.S. Cl. CPC A61B 9/00; A61B 5/1121; A61B 5/1116; A61B 2562/0204; A61B 2562/0219; A61B 2562/0233; A61B 5/1126

(57) ABSTRACT

The present disclosure provides a testing device for testing a subject's susceptibility to injury. The testing device may comprise a base for supporting a frame and an actuator that is supported by the frame, the actuator is configured to impart a physical perturbation upon or proximal a target body-part of the subject. The testing device may also comprise a movement-sensing component for attaching to the tested body-part, the movement-sensing component for detecting the subject's movement after the physical perturbation of the target body-part. The present disclosure also provides a system that includes the testing device and a processor for operating software applications for monitoring and tracking the subject's test data. The present disclosure also provides a method for operating the testing device and system. The present disclosure also provides an apparatus for modifying the stability of a joint of a subject.

### CROSS-REFERENCES TO RELATED APPLICATIONS

This application is a continuation of co-pending International Application No. PCT/CA2018/050211 filed Feb. 23, 2018, which is incorporated herein by reference in its entirety, and additionally claims priority from U.S. Provisional Application No. 62/462,495 filed Feb. 23, 2017, which is incorporated herein by reference in their entirety.

### TECHNICAL FIELD

The present disclosure generally relates to testing a subject's susceptibility to injury. In particular, this disclosure relates to an apparatus, system and method that impart a physical perturbation upon a subject to test for susceptibility to injury. The present disclosure also relates to an apparatus and that provides joint stability.

### BACKGROUND

Humans risk joint and tissue injury during routine physical activities. In particular, impact accidents or participation in sports may put a person at risk of injury due to damaging levels of acceleration or deceleration of the body or a body part. Of particular significance in contact sports are the risks of brain trauma or concussion and injuries to joints. Brain trauma or concussion can occur during an impact to the head, or during an impact to the torso which results in a damaging head motion. Brain trauma or concussion can occur during damaging acceleration, deceleration or rotation of the head. For example, when a sports player is hit by another object, or when a sports player is moving and makes a forceful contact with an object or another person. The relatively soft tissue of the brain means that damaging levels of acceleration, deceleration or rotation can damage brain vasculature, the spinal cord, nerve and cause tissue bruising. Any of these injuries may cause altered brain function with possibly lasting damage.

Brain trauma, including concussion, is additionally challenging because there may be no visible damage following and symptoms of damage may be hard to detect. In the absence of clear symptoms, there are several different types of known assessments for sideline testing or clinical use. Such assessments include assessments of cognitive function, coordination, balance, and eye-movement. Examples of such known assessments include: the Sport Concussion Assessment Tool, the Self-Reported Symptoms Score, the Physical Sign Score, the Glasgow Coma Scale, Maddock's Score for Cognitive Assessment. These tests typically lack baseline data for an individual person, and typically exhibit large individual variation. Test-retest reliability may also be suboptimal for a conclusive diagnosis and/or prognosis.

Sideline assessments and clinical assessments for brain trauma are routinely used to determine if further medical assessments are required, optimal treatments, optimal rest times and conditions or whether a return-to-play is recommended. Accordingly, poor assessment dependability and repeatability can lead to a premature return-to-play recommendation. Return-to-play before a player has fully recovered puts the player at a risk of secondary impact syndrome, which is sustaining another concussion with a significantly lower impact force then the impact that caused the original injury.

Brain trauma prevention-techniques may include the use of protective equipment such as helmets, techniques and training to strengthen certain muscle groups, and removal from play may prevent the incidence of secondary impact syndrome.

It is known that there is significant person-to-person variability in the extent of head motion caused by the same force of impact. This variability may be due to variations in strength, reflex, mass, and anatomy. For example a soccer ball impacting the head may cause significant head motion in a person with a slender neck, while a similar soccer ball impact upon a person with significant neck muscle development is less likely to cause injury. A measurement of neck strength is useful but incomplete because other factors such as stretch reflex and proprioception also influence a person's ability to stabilize their head during an impact.

It may be useful to identify if a person has an elevated risk of injury then specific strengthening and reflex training regimens can be recommended before they take part in a particular sport.

### SUMMARY

Some embodiments of the present disclosure relate to a testing apparatus, which may also be referred to herein as a testing device, for testing a subject's susceptibility to injury. The testing device comprises a base for supporting a frame and an impact component that is supported by the frame. The impact component is configured to impart a physical perturbation upon or proximal to a target body-part of the subject. The testing device also includes one or more movement-sensing components for attaching proximal to a tested body-part. The movement-sensing component is configured to detect movement of the tested body-part after the physical perturbation.

Some embodiments of the present disclosure relate to a testing system for testing a subject's susceptibility to and/or extent of an injury of the tested body-part. The testing system comprises a testing device that comprises a base for supporting a frame and an impact component that is supported by the frame. The impact component is configured to impart a physical perturbation upon or proximal a target body-part of the subject. The testing device also includes a movement-sensing component for attaching proximal to a tested body-part. The movement-sensing component is configured for detecting the subject's movement after the physical perturbation and for creating a movement signal based upon the detected movement. The testing system also comprises a processing structure for receiving the movement signal; and one or more software components that are in operative communication with the processing structure for recording and evaluating the movement signal.

Other embodiments of the present disclosure relate to a method for testing a subject's susceptibility and/or extent of an injury of the tested body-part. The method comprises the steps of: positioning the subject in a first position; imparting a physical perturbation upon a target body-part of the subject; and detecting any movement of a tested body-part following the imparting step.

Without being bound by any particular theory, the embodiments of the present disclosure may allow a subject's baseline susceptibility to an injury to be assessed and then re-assessed following a rehabilitation or training regime or following an injury. The present disclosure may allow a subject's progress through a rehabilitation or training regime to be monitored and/or to assess the extent of the subject's injury in order to assess the subject's readiness to return to play.

The present disclosure also provides an apparatus for modifying the stability of a joint of a subject. The device comprises a flexible planar body with at least one adhesive side that is positionable about one or more joints of a user. The device may also include one or more stiffening elements for at least partially stiffening the flexible planar body while allowing the user a normal range of motion.

### BRIEF DESCRIPTION OF THE DRAWINGS

The features of the present disclosure will become more apparent in the following detailed description in which reference is made to the appended drawings.

FIG. 1 is a side elevation view of a subject being tested with one embodiment of a testing device according to the present disclosure;

FIG. 2 is a perspective view of one embodiment of a frame and a base as part of the testing device shown in FIG. 1;

FIG. 3 is a perspective view of one embodiment of a pneumatic actuator and a height adjustment rod according to the present disclosure;

FIG. 4 is a perspective view of the pneumatic actuator shown in FIG. 3 and one embodiment of a pressure controller according to the present disclosure;

FIG. 5 is a side elevation view of one embodiment of an impact pad according to the present disclosure;

FIG. 6 is a closer view of the pressure controller shown in FIG. 4;

FIG. 7 is a perspective view of one embodiment of a trigger according to the present disclosure;

FIG. 8 is a perspective view of one embodiment of an actuator according to the present disclosure;

FIG. 9 is a front elevation view of one embodiment of a brace device according to the present disclosure for use by a subject that is tested using the testing device according to the present disclosure;

FIG. 10 is a rear elevation view of the brace device shown in FIG. 9;

FIG. 11 is a top plan view of the brace device shown in FIG. 9 in an unfolded position;

FIG. 12 is schematic of a testing system, wherein FIG. 12A shows one embodiment of a testing system according to the present disclosure, FIG. 12B shows another embodiment of a testing system according to the present disclosure, FIG. 12C shows another embodiment of a testing system according to the present disclosure; and FIG. 12D shows another embodiment of a testing system according to the present disclosure; and

FIG. 13 is a schematic of one embodiment of a computing device according to the present disclosure.

### DETAILED DESCRIPTION

Unless defined otherwise, all technical and scientific terms used herein have the same meaning as commonly understood by one of ordinary skill in the art to which this disclosure belongs.

As used herein, the term “about” refers to an approximately +/−10% variation from a given value. It is to be understood that such a variation is always included in any given value provided herein, whether or not it is specifically referred to.

Embodiments of the present disclosure relate to one or more apparatus, system and method for testing a subject's susceptibility to injury. Embodiments of the present disclosure may impart a physical perturbation upon a subject and measure the subject's responsive movements to characterize the stability, susceptibility and/or extent of injury to a specific part of the subject's body. In some embodiments of the present disclosure the subject's responsive movements may identify the subject's susceptibility to injury such as brain trauma, including concussion. In other embodiments of the present disclosure the subject's responsive movements may identify the subject's susceptibility to injury and/or extent of an injury to any one of their tested body-parts including the head, brain, neck, spine, shoulder, elbow, wrist, pelvis, knee or ankle. For clarity, the target body-part is the body part of the subject that is targeted to receive the physical perturbation. The tested body-part is the body part that is coupled to the movement-sensing components for assessing the tested body-part's stability, susceptibility to injury and/or extent of injury. The tested body-part and the targeted body-part may be the same or different.

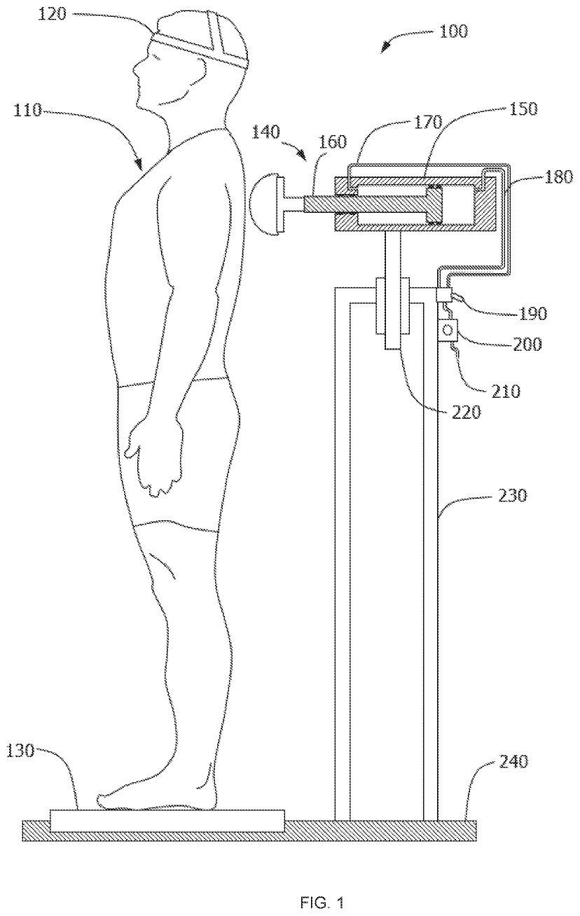

Some embodiments of the present disclosure relate to a testing apparatus, which may also be referred to herein as a testing device 100, for measuring and managing a subject's 110 susceptibility for injury (see FIG. 1). The testing device 100 comprises two primary components: a movement-sensing component 120 and an impact component 140.

The movement-sensing component 120 may be attached to the subject 110 to be tested. The movement-sensing component 120 can be any type of sensor or part of any type of sensor system that detects movement of the subject's 110 body part to which the movement-sensing component 120 is attached, which may also be referred to herein as the tested body-part. When the subject's target body-part and the tested body-part move, the movement-sensing component 120 generates a movement signal that is captured, analyzed and stored, as discussed further below. In some embodiments of the present disclosure, the movement-sensing component 120 may be selected from, but is not limited to, one or more: accelerometers, infrared sensors, optical sensors, laser trip sensors, time-of-flight laser sensors, vibration sensors, acoustic sensors, video movement sensors or combinations thereof. Some embodiments of the present disclosure relate to the testing device 100 that includes a plurality of movement-sensing components 120 that are positioned on different parts of the subject's 110 body, such as the head, the chest, the pelvis, other body parts or combinations thereof.

FIG. 1 shows one embodiment of the movement-sensing component 120 that is attached to the subject's 110 head for testing the subject's 110 head for injury, trauma and/or concussion susceptibility. In this instance the head is being tested and is referred to as the tested body-part. This embodiment of the movement-sensing component 120 may be a cap or headband that fits snuggly and securely to the subject's 110 head. The movement-sensing component 120 securely houses motion-sensors such as one or more accelerometers. In another embodiment, the movement-sensing component 120 is optical-based and the cap or headband may include optical markers that can be tracked by one or more external cameras (not shown). The cap or headband may include marks to aid in consistent positioning so that, for example, one sensor may be consistently positioned on the subject's head.

It is understood that if other tested body-parts of the subject 110 are being tested for susceptibility to injury, the movement-sensing component 120 may be positioned upon or about a different tested body-part than the head. For example, the tested body-part may be a joint and the movement-sensing component 120 may be positioned at a target body-part above, below or both of the tested body-part joint of the subject 110.

The impact component 140 is capable of moving and imparting a rapid impact upon the subject 110, referred to herein as a physical perturbation. The amplitude of the physical perturbation is predetermined and/or calculated so as not to cause any injury to the subject 110. In some embodiments of the present disclosure, the impact component 140 comprises an actuator 150 that is configured to move an actuator arm 160. The actuator arm 160 may include a soft impact pad 260. The actuator 150 moves the actuator arm 160 by one or more movement system such as: a pneumatic system, a hydraulic system, an electric motor, a linkage to a gravity drop-tower, a pendulum, a spring, an elastic element, an electromagnetic motor or combinations thereof.

The subject 110 may stand still while the actuator arm 160 imparts the physical perturbation upon the subject 110. The amplitude of the physical perturbation can be controlled and adjusted so that a desired force of the physical perturbation is applied to the subject 110. For example, when the actuator 160 is pneumatic a supply of pressurized gas can be adjusted to increase or decrease the amplitude of the physical perturbation. In some embodiments of the present disclosure the amplitude of the physical perturbation applied to the subject 110 can be in a range of about 0.01 joules (J) to about 75 J. In other embodiments of the present disclosure the amplitude of the physical perturbation applied to the subject 110 can be in a range of about 0.01 joules (J) to about 69 J. In some embodiments of the present disclosure, the operator may select the amplitude of the physical force based upon the subject's 110 age, weight, physical strength, neck circumference, neck length, level of training and the specific tested body-part and target body-part for a given test.

Following the physical perturbation, the subject's 110 reflexive or resulting movement causes the movement-sensing component 120 to generate the movement signal. The extent of the subject's 110 movement, as reflected by the movement signal, is used to determine the subject's 110 risk of injury and/or risk of re-injury of the tested body-part. Optionally, the subject 110 may also wear protective equipment during the testing such as a neck stiffening guard or a helmet.

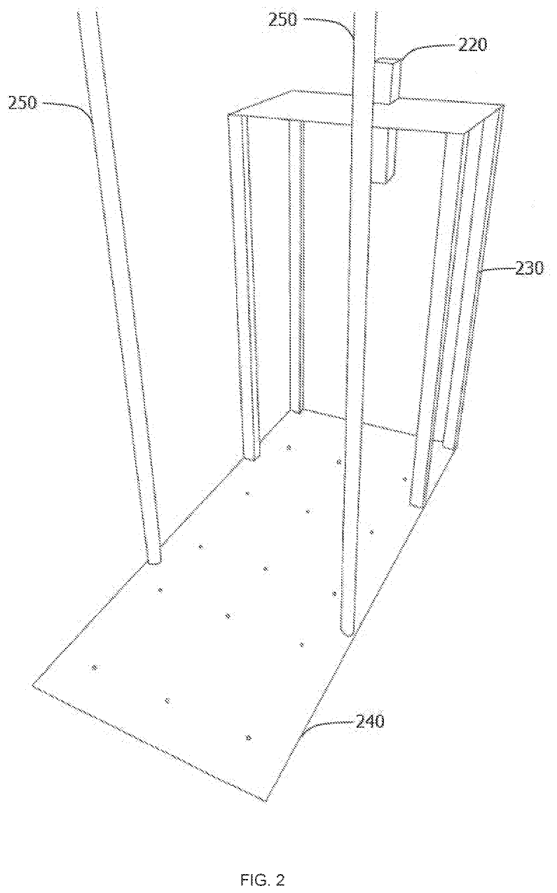

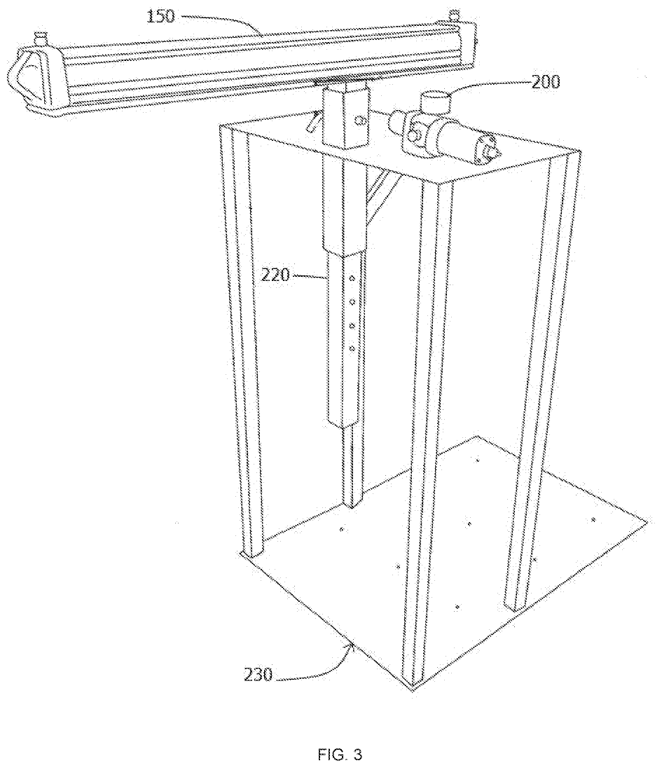

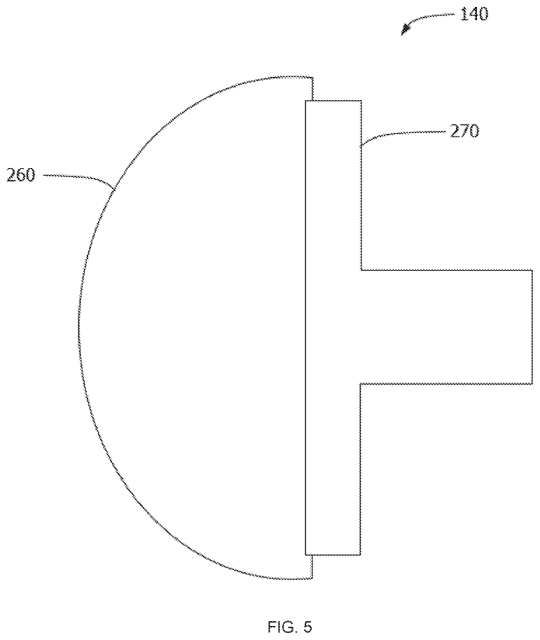

FIG. 1 and FIG. 2 show other features of the testing device 100 such as a base 240 which supports a frame 230. FIG. 1 shows the subject 110 standing on an optional weigh scale or force plate 130 of the testing device 100, the force plate 130 is also supported by the base 240. The subject 110 has a headband which incorporates accelerometers as part of the testing device's 100 movement-sensing component 120. The subject 110 is positioned to receive a physical perturbation to their back. In some embodiments of the present disclosure, the impact component 140 has an impact pad 260 connected to the actuator arm 160 via a stiff backing plate 270 (see FIG. 5). The impact pad 260 may comprise a soft, flexible, or compressible material so as to avoid injuring the subject 110 when the actuator arm 160 imparts the physical perturbation. The actuator 150 may be attached to a frame 230, via a height-adjustment system 220, such that the vertical position of impact component 140 can be adjusted relative to the base 240 so that the position of where the physical perturbation is imparted upon the subject 110 can be adjusted (see FIG. 3).

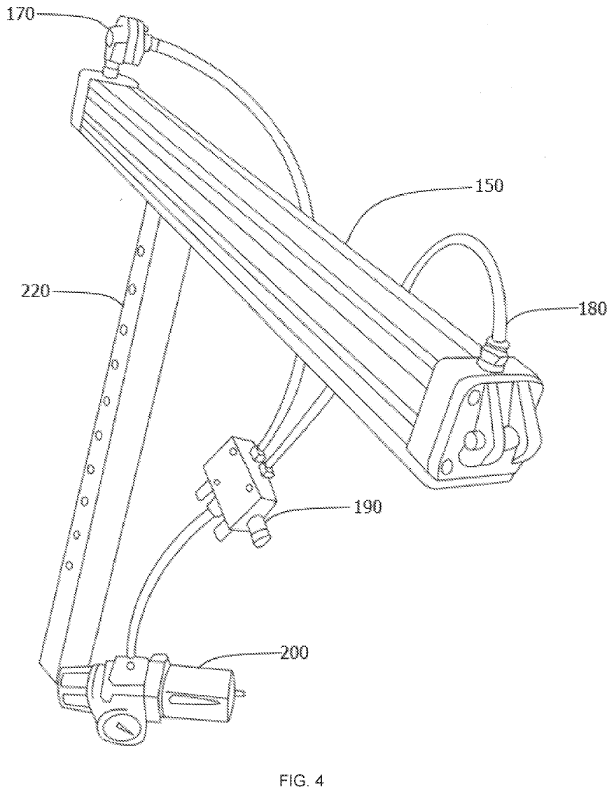

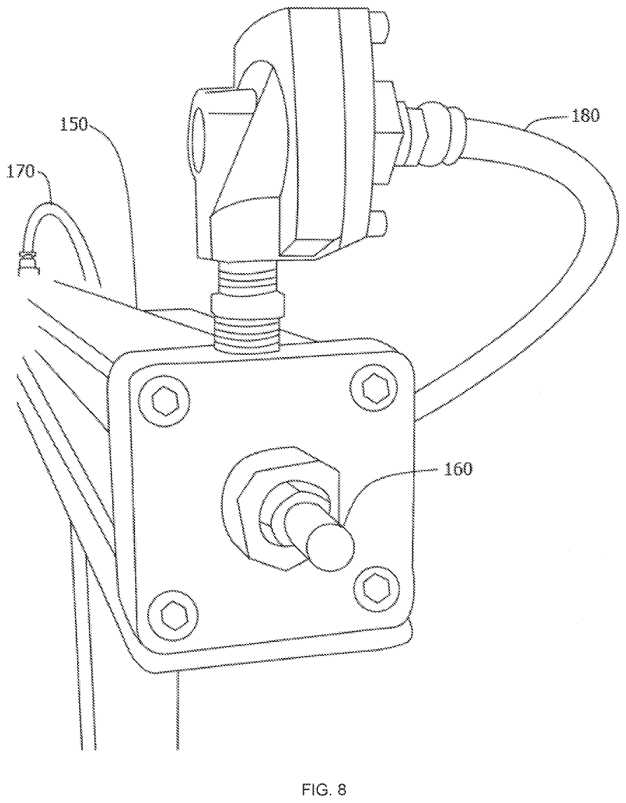

In some embodiments of the present disclosure, the actuator 150 is pneumatic and the actuator arm 160 includes a piston within a cylinder. Increasing the fluid pressure on either side of the piston, within the cylinder, accelerates the actuator arm 160 in a first direction or a second direction. The fluid pressure is modulated by pressurized air that is supplied into the cylinder through flexible compressed air lines 170, 180 that are connectible at either end of the actuator 150 (see also FIG. 4 and FIG. 8).

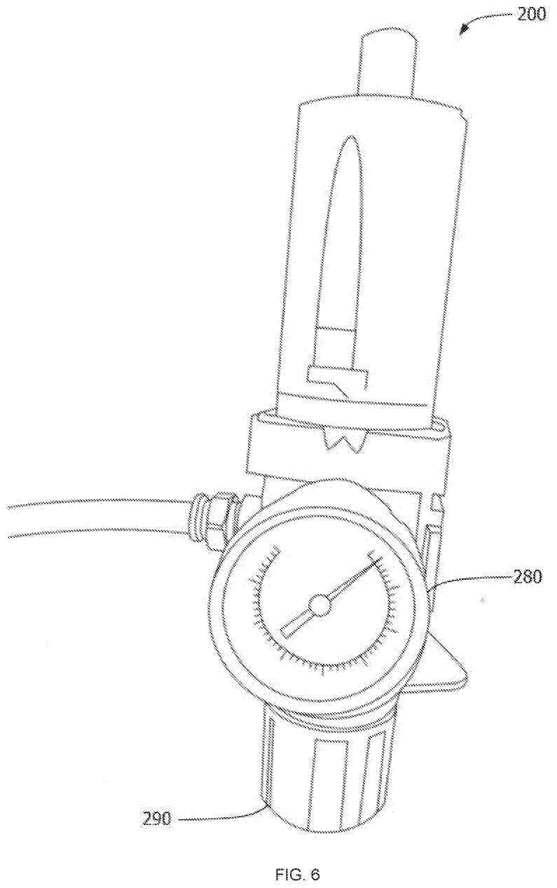

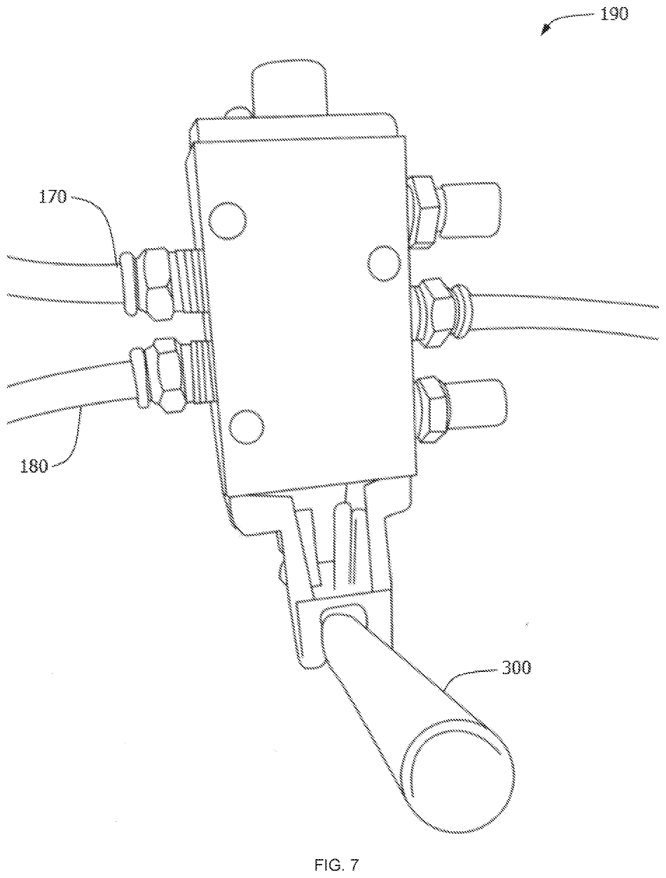

As shown in FIG. 6, an operator may control and trigger the actuator 150 manually by using a pressure controller 200 that includes a pressure gauge 280, a pressure modulator 290 and a trigger 300. The trigger 300 is shown most clearly in FIG. 7. Alternatively, the actuator 150 may be controlled by a testing system 400 (described further below).

FIG. 2 shows optional stance position marker members 250 that are configured to aid with consistent positioning of the subject 110 relative to the impact component 140. For clarity, the actuator 150 is not shown in FIG. 2.

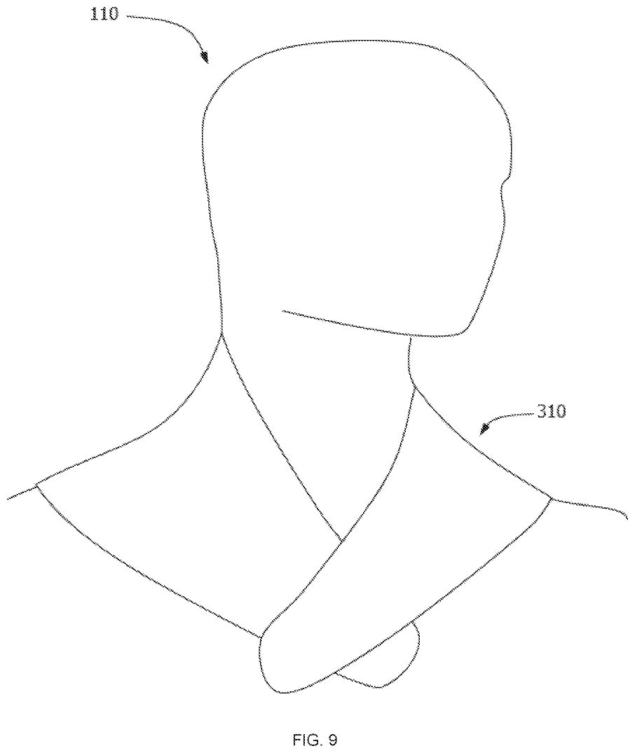

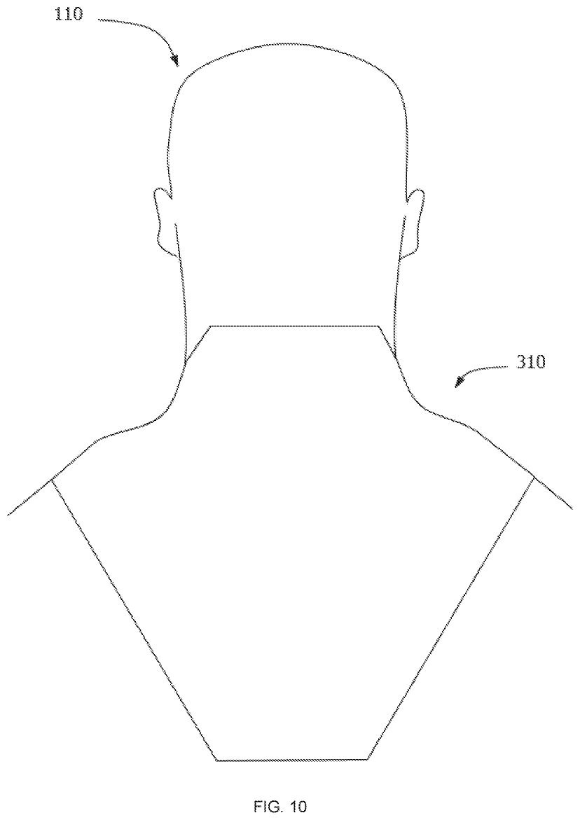

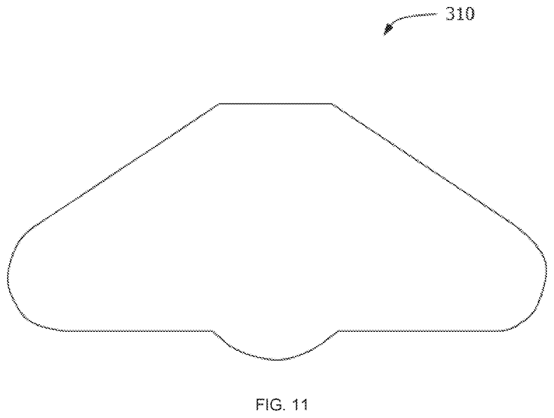

Some embodiments of the present disclosure relate to a neck guard 310 (see FIG. 9, FIG. 10 and FIG. 11). The neck guard 310 may be a generally planar body made from a flexible or semi-flexible fabric sheet. The neck guard 310 may have at least one side that includes an adhesive element for temporarily securing the neck guard 310 to the user's skin or for securing other components on to the neck guard 310. The neck guard 310 may be stretched and stuck to the subject's back, the back of the neck, the sides of the neck, and chest. When attached, the neck guard 310 may provide additional stiffness to the head-neck segment of the subject 110. In some embodiments of the present disclosure the neck guard 310 may include one or more stiffening elements, such as viscoelastic or thixotropic elements for example a sealed flexible bladder containing a non-Newtonian liquid so that the neck guard 310 can become stiffer in the milliseconds following an physical perturbation or other type of impact. However, prior to any physical perturbation or other type of impact, the stiffening elements are not activated and the neck guard is relatively flexible. The neck guard shape and flexibility is such that normal, non-injury motion of the head-neck joint is still possible and the neck guard is suitable for wearing during play. Additionally, the tension created upon the wearer's skin may improve the subject's 110 physiological stabilization of the head-neck segment by providing a heightened proprioceptive effect or awareness. Different sizes and stiffness of neck guard 310 can be used to provide a good fit and to match the neck stabilization needs of different subjects 110. Optionally, the neck guard 310 may include ventilation holes or ventilation pores for moisture and heat management, or to allow more fabric stretch in areas of high curvature for better form fitting.

Some embodiments of the present disclosure relate to the testing system 40 that comprises the testing device 100 and at least one computing device that is in operative communication with at least the movement-sensing component 120 of the testing device 100. Optionally, at least one computing device is also in operative communication with the impact component 140 for controlling the timing and amplitude of the physical perturbation that is imposed upon the subject 110.

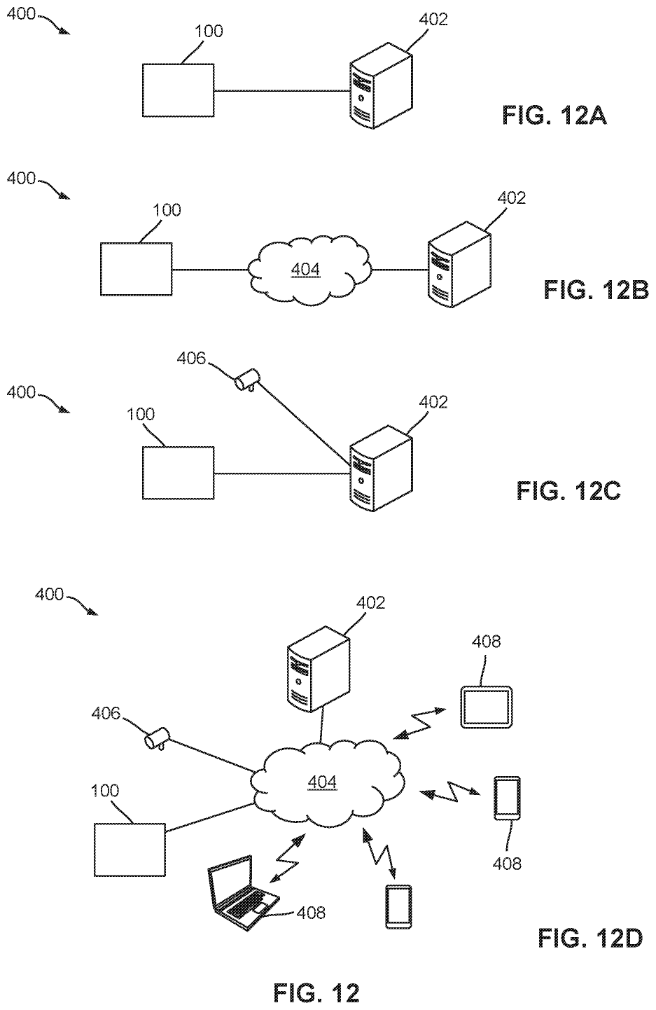

For example, as shown in FIG. 12A a testing system 400 comprises the testing device 100 and a testing computing device 402 in operative communication with each other via suitable wired communication such as a USB cable, a serial communication cable, a parallel communication cable, wireless communication, such as WI-FI® (WI-FI is a registered trademark of the City of Atlanta DBA Hartsfield-Jackson Atlanta International Airport Municipal Corp., Atlanta, Ga., USA), BLUETOOTH® (BLUETOOTH is a registered trademark of Bluetooth Sig Inc., Kirkland, Wash., USA), ZIGBEE® (ZIGBEE is a registered trademark of ZigBee Alliance Corp., San Ramon, Calif., USA), and/or the like.

As shown in FIG. 12B, the testing system 400 may be a cloud-based system in which the testing device 100 and the testing computing device 402 are in communication with each other via a network 404 such as Ethernet, Internet, 3G/4G/5G wireless mobile telecommunications network, and/or the like via suitable wired and/or wireless communication means.

As shown in FIG. 12C and FIG. 12D, in some embodiments, the testing system 400 may further comprise a camera 406 in operative communication with the testing computing device 402 for obtaining one or more visual cues of the subject 110. In the example shown in FIG. 12C, the camera 406 is in operative communication with the testing computing device 402. In the example shown in FIG. 12D, the camera 406 is in operative communication with the testing computing device 402 via the network 404. Also shown in the non-limiting embodiment of FIG. 12D, the testing system 400 may also comprise one or more client computing devices 408 in operative communication with the testing computing device 402 for inputting user instructions to the testing computing device 402 and for receiving data such as testing results from the testing computing device 402. The client computing devices may be any one or more of desktop computers, laptop computers, tablets, smartphones, personal digital assistants (PDAs), and the like.

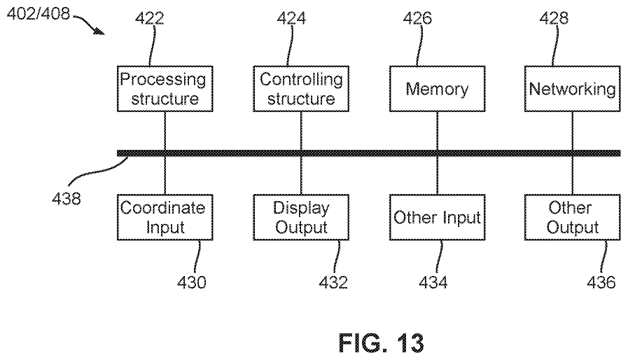

In some embodiments of the present disclosure, the testing computing device 402 and the client computing device 408 may have a similar hardware structure such as a hardware structure 420 shown in FIG. 13. The computing devices 402/408 can each comprise a processing structure 422, a controlling structure 424, memory or storage 426, a networking interface 428, a coordinate input 430, display output 432, and other input and output modules 434 and 436, all of which are operatively interconnected by a system bus 438.

The processing structure 422 may be one or more single-core or multiple-core computing processors such as INTEL® microprocessors (INTEL is a registered trademark of Intel Corp., Santa Clara, Calif., USA), AMD® microprocessors (AMD is a registered trademark of Advanced Micro Devices Inc., Sunnyvale, Calif., USA), ARM® microprocessors (ARM is a registered trademark of Arm Ltd., Cambridge, UK) manufactured by a variety of manufactures such as Qualcomm of San Diego, Calif., USA, under the ARM® architecture, or the like.

The controlling structure 424 comprises a plurality of controllers, such as graphic controllers, input/output chipsets and the like, for coordinating operations of various hardware components and modules of the computing devices 402/408.

The memory 426 comprises a plurality of memory units accessible by the processing structure 422 and the controlling structure 424 for reading and/or storing data, including input data and data generated by the processing structure 422 and the controlling structure 424. The memory 426 may be volatile and/or non-volatile, non-removable or removable memory such as RAM, ROM, EEPROM, solid-state memory, hard disks, CD, DVD, flash memory, or the like.

The networking interface 428 comprises one or more networking modules for connecting to other computing devices or networks through the network 404 by using suitable wired or wireless communication technologies such as Ethernet, WI-FI, BLUETOOTH®, ZIGBEE®, 3G/4G/5G wireless mobile telecommunications technologies, and/or the like.

The display output 432 comprises one or more display modules such as monitors, LCD displays, LED displays, projectors, and the like, for providing a user interface (UI). The display output 432 may be a physically integrated part of the computing device 402/408 (for example, the display of a laptop computer or tablet), or may be a display device physically separate from, but functionally coupled to, other components of the computing device 402/408 (for example, the monitor of a desktop computer).

The coordinate input 430 comprises one or more input modules for one or more users to input coordinate data, such as touch-sensitive screen, touch-sensitive whiteboard, trackball, computer mouse, touch-pad, or other human interface devices (HID) and the like. The coordinate input 430 may be a physically integrated part of the computing device 402/408 (for example, the touch-pad of a laptop computer or the touch-sensitive screen of a tablet), or may be a display device physically separate from, but functionally coupled to, other components of the computing device 402/408 (for example, a computer mouse). The coordinate input 430, in some implementation, may be integrated with the display output 432 to form a touch-sensitive screen or touch-sensitive whiteboard.

The computing device 402/408 may also comprise one or more other inputs 434 such as keyboards, microphones, scanners, cameras, and the like. The computing device 402/408 may further comprise other outputs 436 such as speakers, printers and the like.

The system bus 438 interconnects various components 422 to 436 enabling them to transmit and receive data and control signals to/from each other.

The testing computing device 402 comprises and executes one or more of the following supporting software-components: a management and tracking software; an administrator interface; an optional eye-tracking software; an optional sideline testing application; an optional reaction test; an optional educational system for neck strength training and conditioning. In some embodiments, some of the supporting software-components may be stored in and executed by one or more client computing devices 408.

The processing structure 422 may receive the movement signal from the movement-sensing component 120 and the memory 426 may save the movement signal received from each test. The testing system may operate the software components, which in turn may control one or more of: the height position of the actuator 150, the amplitude of the physical perturbation; and recording and assessing all movement signals received to determine whether a test should be repeated.

Without being bound by any particular theory, the subject 110 may demonstrate a reflexive muscle-contraction if the subject 110 anticipates the physical perturbation. Anticipatory reflexive muscle-contractions may interfere with the accuracy and consistency of the test results. The testing device 100 may also include further components that promote or suppress any anticipatory reflexive muscle-contraction. Components that may promote the reflexive contraction may occur by an audible alert such as a buzzer which occurs momentarily before the impact, for example 0.5 seconds before the impact which is sufficient time for a reflexive contraction. The reflexive muscle contraction may be suppressed by components that ensure a quiet operation of the actuator 150, sound suppression or sound masking, use of a blindfold. The reflexive muscle contraction may also be suppressed by ensuring that the physical perturbation is short and rapid for example less than 0.1 second long, which is generally less time than a human reaction time in order to cause a muscular contraction.

Optionally, the subject 110 may be tested while wearing additional equipment. This may include the neck guard 310 to provide additional stiffening of the subject's neck. The additional mass of helmets may exacerbate or attenuate the head movement resulting from a physical perturbation of the subject's 110 torso. The subject 110 may also wear a helmet to better characterize their on-field head movement risk. Other equipment such as shoulder pads may also be used to measure their effect on head movement.

The testing device 100 and testing system 400 may also include any of the following safety features: a limit on the total amplitude of the physical perturbation; a pressure limit and size limit for the actuator 150 such that the total amplitude of the physical perturbation is below an injury threshold for even the smallest subject 110 who will be tested with the testing device 100; training and certification of operators; access codes which preclude unauthorized use; one or more impact targeting members including one of a laser guide, a light beam guide, a view finder guide, a locator guide for the subject 110 or combinations thereof such that the location of the physical perturbation can be accurately targeted and off-target impacts are minimized; integration of the weight scale 130 with the system's software to limits the physical perturbation energy as a function of the subject's 110 weight; a breakaway element between the actuator 150 and the impact pad 260 that will break, fail, or buckle if the forces exceed a predetermined threshold; a mechanical stop on the actuator 150, such that motion does not exceed a safe limit or combinations thereof.

The testing device 100 and the testing system 400 can be used as follows. The subject 110 assumes a position adjacent to the impact component 140. The impact component 140 is then triggered to move and impart a small but rapid physical perturbation upon the subject 110. The testing device 100 and the testing system 400 may be suitable for screening healthy subjects before they start a playing season and before they experience an on-field impact.

Personal data for each athlete is entered into the management and tracking software for example: name, gender, age, height, weight, sport and level.

The subject 110 straps the movement-sensing component 120 on their head (or other target body-part) and steps onto the base 240 and assumes a first position of six positions. The height of the impact pad 260 is adjusted so the top of the impact pad 260 is aligned with the subject's 110 suprascapular border or the subject's 110 torso.

In embodiments of the actuator 150 that are pneumatically powered and controlled, the air pressure (for example in pounds per square inch, psi) is adjusted to the desired amplitude of the physical perturbation range for the subject 110 based on a predetermined chart using the pressure controller 200. Optionally, this step may be performed automatically by the management and tracking software of the testing system 100.

The operator can use the trigger 300 to move the actuator arm 160 to impart the physical perturbation of the subject 110. The physical perturbation can cause the subject 110 to brace upon impact. The biomechanical axis of angular acceleration and the rate of acceleration of the subject's 110 head and neck, as well as the G-forces created are detected by the movement-sensing component 120 and recorded by the testing system 400.

The testing system 400 may then prompt the operator to save the test data or to retest the subject 110, or these steps may be performed automatically by the testing system 400. Once the data is saved it is transmitted in real-time to the management and tracking software. Optionally, the management and tracking software may be cloud-based. The subject 110 then proceeds through any remaining positions to complete the test. The positions are chosen to isolate and characterized different movements caused by various impacts, including but not limited to: a frontal impact on the chest midline parallel to a sagittal axis to measure neck flexion; an impact on the back midline parallel to a sagittal axis to measure neck extension; an impact on the back right of midline parallel to a sagittal axis to measure neck rotation to the right; an impact on the back left of midline parallel to a sagittal axis to measure neck rotation to the left; an impact on the right shoulder parallel to a frontal axis to measure neck bend to the right; and an impact on the left shoulder parallel to a frontal axis to measure neck bend to the left. Optionally other positions may include kneeling or seated.

At this point, the subject 110 steps off the testing device 100 and stands in a resting position where they are motionless with their feet together and arms crossed while wearing the movement-sensing apparatus 120 for about 20 seconds. The testing system 400 may also provide a visual cue to the operator that the resting position data has been saved and transmitted.

The testing system 400 can then compare the movement signal obtained following the physical perturbation when the subject 100 was in all positions, including the resting position and compares those to a predetermined chart of comparators. The output of the testing may be a quantification of the subject's neck stability (or the stability of another target body-part). If this is outside a normal stability range, then a no-play recommendation may be generated together with a rehabilitation or training regimen. Subsequent tests may be performed on the subject 110 to confirm if the subject's neck stability has improved and to assess the subject's readiness to return to play.

The subject 110 can be re-tested and the results before and after the rehabilitation or training regimen can be compared. In some instances, the subject 110 may be tested every 12 weeks in the initial stages, and less frequently as they begin to reach higher levels of performance on the testing.

I claim:

1. A testing device for testing a subject's susceptibility to injury, the testing device comprises:
a) a base for supporting a frame;
b) an impact component that is supported by the frame, the impact component is configured to impart a physical perturbation upon or proximal to a target body-part of the subject; and
c) one or more movement-sensing components for attaching proximal to a tested body-part, the movement-sensing component is configured to detect movement of the tested body-part after the physical perturbation.

2. The testing device of claim 1, wherein the impact component comprises an actuator arm for imparting the physical perturbation.

3. The testing device of claim 2, wherein the actuator arm further comprises an impact pad.

4. The testing device of claim 2, further comprising a movement system for moving the actuator arm, wherein the movement system is a pneumatic system, a hydraulic system, an electric motor, a linkage to a gravity drop-tower, a pendulum, a spring, an elastic element, an electromagnetic motor and combinations thereof.

5. The testing device of claim 1, wherein the one or more movement-sensing components are one or more accelerometers, infrared sensors, optical sensors, laser trip sensors, time-of-flight laser sensors, vibration sensors, acoustic sensors, video movement sensors and combinations thereof.

6. The testing device of claim 1, further comprising a force plate that is supported by the base.

7. The testing device of claim 1, further comprising one or more stance position members for assisting with positioning a subject relative to the actuator.

8. The testing device of claim 1, further comprising a height-adjustment system for adjusting a height of the impact component relative to the base.

9. The testing device of claim 1, further comprising a component that promotes an anticipatory reflexive muscle-contraction of the subject.

10. The testing device of claim 1, further comprising a component that suppresses an anticipatory reflexive muscle-contraction of the subject.

11. The testing device of claim 1, further comprising one or more impact targeting members.

12. The testing device of claim 1, wherein the physical perturbation has an amplitude of between about 0.01 joules (J) and about 75 J.

13. A testing system for testing a subject's susceptibility to injury, the testing system comprising:
a) a testing device that comprises:
i) a base for supporting a frame;
ii) an impact component that is supported by the frame, the impact component is configured to impart a physical perturbation upon or proximal a target body-part of the subject; and
iii) a movement-sensing component for attaching proximal to a tested body-part, the movement-sensing component is configured for detecting the subject's movement after the physical perturbation and for creating a movement signal based upon the detected movement;
b) a processing structure for receiving the movement signal; and
c) one or more software components that are in operative communication with the processing structure for recording and evaluating the movement signal.

14. A method for testing a subject's susceptibility to injury, the method comprising steps of:
a) positioning the subject in a first position;
b) imparting a physical perturbation upon a target body-part of the subject; and
c) detecting any movement of a tested body-part following the imparting step.

15. The method of claim 14, further comprising a step of detecting any further movement after moving the subject to a resting position.

16. The method of claim 14, further comprising a step of recording the detected movement of the tested body-part.

17. The method of claim 14, wherein the tested body-part is one or more of the subject's head, neck, spine, shoulder, elbow, wrist, pelvis, knee and ankle.

18. The method of claim 14, further comprising a step of applying a neck guard to the subject's neck.

19. The method of claim 14, wherein the tested body-part is the subject's head and the target body-part is the subject's suprascapular border.

20. The method of claim 14, wherein the tested body-part is the subject's head and the target body-part is the subject's torso.

21. The method of claim 14, wherein the physical perturbation has an amplitude of between about 0.01 joules (J) and about 75 J.

22. A joint-stability device comprising:
a) a flexible planar body with at least one adhesive side that is positionable about one or more joints of a user; and
b) one or more stiffening elements for at least partially stiffening the flexible planar body while allowing the user a normal range of motion.
